(19) United States
(12) Patent Application Publication
(10) Pub. No.: US 2016/0144140 A1
(43) Pub. Date: May 26, 2016

(54) DISPOSABLE-INHALER FOR SUBSTANCES IN POWDER FORM

(71) Applicant: SIMPLIFIED SOLUTIONS SWEDEN AB, Lindome (SE)

(72) Inventors: Jan Åberg, Kållered (SE); Yutaka Kataoka, Lindome (SE); Jan Hedegaard-Broch, Gzira (MT); Stefan Fransson, Göteberg (SE); Ulf Rytterholm, Bålsta (SE)

(21) Appl. No.: 14/786,861

(22) PCT Filed: Apr. 23, 2014
(86) PCT No.: PCT/SE2014/050489

§ 371 Date: Oct. 23, 2015

(30) Foreign Application Priority Data
Apr. 23, 2013 (SE) 1330041.3
Jul. 11, 2013 (SE) 1300485-8

(51) Int. Cl. A61M 15/00

(52) U.S. Cl. CPC A61M 15/0051; A61M 2202/064

(57) ABSTRACT

The invention relates to a disposable inhaler intended for single use and for substances in powder form which comprises a preferably elongated shape body arranged to, at least partially be inserted in the user's mouth, including one or more air channels arranged in the shape of the body, that a foil is arranged with inlet holes and an outlet holes in connection with the air channels, that a substance in powder form is placed in the air channels, that the foil is arranged to, in the inhaler storage mode, cover the inlet and outlet holes and thus prevent the substance in powder form to fall out of the inhaler air channels, and that tape is removable to be able to use the inhaler and when it is removed exposes the inhaler in-and outlet holes and thus facilitates that the substance in powder form, which is placed in the air channel, can leave the air channel using the airflow that occurs when the user inhales, through the inhaler. The invention is achieved through that the air channel is arranged with a canted end-wall near the outlet hole, the canted end-wall is helping to angle the airstream out of the air channel obliquely upward at its outlet.

### TECHNICAL FIELD

This invention relates generally to disposable inhalers for substances in powder form intended for inhalation. Specifically it refers to a so-called DPI, “Dry Powder Inhaler” operated by the user's own respiration. Single-dose inhalers are as multidose inhalers, designed to alleviate disease caused by asthma or other problems that prevent normal respiration. New applications that are especially suitable for single-dose disposable inhalers are the relief of migraine, flu vaccination, etc.

### TECHNICAL BACKGROUND

On the market today there are a large number of different inhalers for substances in powder form, most of which are so-called multi-dose inhalers. The inhalers currently marketed are relatively complex in their design. Known disposable inhalers consist of relatively many parts which makes them complicated and thus expensive to manufacture and also expensive to buy for the end user.

A large proportion of asthmatics have less severe problems, for example, only at high pollen levels or on occasional contact with animal fur. These patients may need to make only a few inhalations per year, hence, a disposable inhaler is a much more economical option than a multidose inhaler. In poorer countries, such as in the Third World, tablets are often sold one by one to customers cannot afford to buy more than one at a time. The cost is crucial and multi-dose inhalers are too expensive. Migraine medication is another application for inhalers since the therapeutic effect is achieved significantly faster through the lungs. A tablet must first be dissolved in the gastric content and then pass out through the stomach wall to reach the bloodstream.

At vaccination the drug/syringe must always be managed by a registered nurse. The shortage of registered nurses in developing countries is large and disposable syringes are not cheap. An instructor with no medical training can, using the present invention, instruct users how to perform the inhalation, thus one becomes independent of the shortage of nurses for treatment.

Most of the disposable inhalers on the market today, such as the Boeringer Ingelheim Handihaler® and Aptars Twister®, consist of one inhalation device that adds a capsule that is punctured after which the user inhales the dose. The way the capsule is punctuated carries a risk that the dose in the capsule may become contaminated by the punctured tool. In addition, the actual device in which the capsule is added to be punctured is relatively expensive to manufacture. The present invention eliminates the expensive injection molded plastic inhalation device, which greatly reduces the cost of production and also removes the risk of contaminating the dose. Disposable inhalers exists that are patented (but not yet on the market), such as, for example, U.S. Pat. No. 6,286,507B1, U.S. Pat. No. 6,105,574, US20130025593A1 and WO2012/004485. They are all relatively expensive to manufacture and/or difficult to manage or have one, for the end-user, unsafe construction. U.S. Pat. No. 6,286,507B1 and U.S. Pat. No. 6,105,574 show two different single dose disposable inhalers where an upper part and a lower part are assembled and in between them form an air channel. In the lower part there is a recess for positioning of the medical powder. The powder is contained in the recess with the use of a tape. A portion of the tape is sticking out of one end of the air channel. The user can grab the protruding part of the tape and pull it from the recess and thus expose the dose for inhalation. U.S. Pat. No. 6,286,507B1 describes specifically one alternative design where a flat metal part is arranged with a recess for the powder. The recess has a hole at the bottom and two parts of tape encloses the powder, a tape from the top, and another tape from below. The two parts of the tape are merged so that the user can grab and remove them in a single operation exposing the powder. U.S. Pat. No. 6,105,574 describes specifically that a deformation in the lower part should contact the user's lower lip causing the air outlet channel to be placed approximately 30 mm into the oral cavity. That makes the air outlet channel to a higher degree, to be located on top of the tongue instead of air beam hitting straight on your tongue which is common in traditional inhalers. The amount of powder that sticks on the tip of the tongue is reduced accordingly. The powder that sticks in the cavity of the mouth is swallowed to a high degree with the saliva and hence its clinical effect is lost. The method to enclose the powder in a recess inside the air channel has the disadvantage that the air channel is open. This means that foreign objects, particles from the contents of pockets or purses where the inhaler is stored, falls into the air channel. Since it is medically unacceptable for foreign particles to be inhaled, the air channel must be protected in an appropriate manner. Inhalers must be approved by the authorities and an open air channel is not accepted. All inhalers have a cap or a similar protection to prevent the ingress of foreign particles. Some inhalers, for example, described in U.S. Pat. No. 6,286,507B1 and U.S. Pat. No. 6,105,574, must have a cap at each end of the air channel, or a sealed bag that is torn up right before inhalation. The cap or the bag is an additional cost which in this context is of certain importance. It also requires an additional operation which the user has to perform.

The present invention has at least one closed air channel, a single tape that seals the intake holes, vent holes, at least one air channel and at least one recess for the medical powder. Everything is sealed by means of one tape. Foreign particles cannot enter the inhaler and consequently there is no need for protective caps or bags at the same time as the operation of the inhaler will be easier.

US20130025593 Describes a single dose inhaler wherein a capsule is to be positioned and then punctured prior to inhalation. The design consists of several parts, and the user must perform several steps to be able to take the intended dose. Contamination of the dose is also an obvious risk in this construction.

WO2012/004485 describes a single dose inhaler where a dose of drug in a blister pack is placed in an injection molded inhaler which is then sealed. The blister is opened by having a strip from the same blister protruding a bit from the inhaler after the inhaler is loaded with one dose. By pulling the strip the dose inside the blister is opened into the inhaler and can then be inhaled. This solution involves an increase in the cost of production compared with the present invention because an injection molded inhaler must be used. In addition, the end user performs more steps than in the use of the present invention before the patient/user can take the dose. This is because the inhaler must be opened, a blister capsule has to be added, the inhaler must be closed, the dose exposed by dragging the protruding part of the blister and then to be brought to the mouth for inhalation.

### SUMMARY OF THE INVENTION

For the purpose of simplicity the following summary of the invention will describe a disposable inhaler with at least two separate recesses for medical powder. The same concept can be used with just one recess which is described in the “Description of embodiments” section, FIGS. 6A to 10B.

The disposable inhaler consists of, according to the invention, a body with a lower part/shaped body comprising at least one powder chamber in form of recesses. The recesses are also the disposable inhaler air channels and can simultaneously be its powder chambers. The air channels are covered by a foil provided with at least one hole. The holes allow air to pass through the air channels and the medical powder is emptied when the user inhales. The first series of holes thus represents the inlet holes and a second series of holes are the outlet holes. The foil can preferably be transparent. The powder in the air channels hence becomes visible to the user. After inhalation, the user can visually inspect that all the powder has been used. The foil is covered by a sealing tape. The tape is equipped with adhesive or warm formed material in the area between the holes and around them. One of the ends of the tape is devoid of adhesive material. The users can, with their fingers grasp this part of the tape and pull it off in its entirety thus freeing the inlet and outlet holes from their in-closure. The users should keep the one-time inhaler substantially horizontally when the tape is pulled away so that the powder in the powder chambers does not run the risk of being spilled out. The inhalers' predominantly horizontal position should also be used when inhaling. An instruction for the handling of the one-time-inhaler is appended or indicated on the inhaler. The user inserts the inhalers' allotted end in his mouth, takes a deep breath through the inhaler and inhalation is thus completed after which the exhausted inhaler can be disposed.

The purpose of the invention is to provide an as cheap disposable inhaler, as possible, which also has at least one powder chamber. The use of two or more powder chambers is necessary since sometimes the active ingredients to be used might react with each other if mixed in one chamber and hence loses the medical effect for the patient i.e. the ingredients may only be mixed with each other when in the body of the patient i.e. at inhalation in this case. The innovative inhaler is preferably constructed as a blister, a technique which is common for packing various products, toys, etc. It is also common that medical tablets are packaged in blisters. A blister is a thermoformed transparent or molded part, a lower part, which, for example, forms the space for tablets. The upper part is mostly quite flat and generally has a descriptive text printed on the surface. There are blisters where both the lower-and upper parts are thermoformed, however, that means that the cost increases. The process of pressing the text is complicated and more costly. The technology to produce blister is proven since decades, the cost is very low in this context. Blisters with a thermoformed transparent or molded underside and a flat top dominate the packaging market for objects under 20 cm in size. The large industrial base with gigantic volumes and competition between many vendors with very trimmed production processes, provide very low costs. Another purpose of the invention is that it is designed in such a way that the user can conveniently store a number of disposable inhalers in their purse or pocket. A disposable inhaler in the form of a blister might for example have a width 18 mm and a length of 75 mm (when two separate chambers are used per inhaler). It is usual for blisters to be attached to each other and being able to be torn apart in a perforation. Similarly, the disposable inhalers under the invention can be put together in a map of e.g. three one-time-inhalers with an overall dimension 54×75 mm (again if two separate chambers are used per inhaler). The user can tear off one inhaler at a time. The three disposable inhalers may, with a suggested dimension of 60×75 mm, comfortably be stored, for example in a breast pocket. The thickness is only 4-5 mm which is an advantage in terms of comfort. Length and width are about the credit-card size, also a comfort factor for the customers.

Another purpose of the invention is that the handling of the disposable inhaler should be simple and user-friendly. When the user has pulled off the tape the powder in the powder chambers can be made visible, given that the foil is transparent. The powder is usually of a light color or white and preferably, the lower part of the inhaler can be designed, in part, with a dark color to create contrast. After inhalation, the user can visually check that the powder has been consumed and that it therefore followed with the inhaled-airflow. Hence, the user can visually see that the inhalation of all of the powder has been successful. The construction also entails that the user does not need to open a protective bag around the disposable inhaler. Other designs have an open air channel which could cause contamination from the handbag or pocket interior into the air channel and by inhalation, down in the lungs. This is medically unacceptable; a protective bag must protect the air channel from contamination. A protective bag is an additional cost and brings additional steps that the user must carry out. The inventive disposable inhaler is its own protective bag. The tape seals the air channels off at both ends, no contamination can occur and no foreign particles can get into the same. After the doses are inhaled the inhaler is discarded.

A unique feature is hence achieved through letting the inhaler have at least two separate powder chambers whose content is inhaled simultaneously.

Yet another unique feature is to let the inlet and outlet holes for the air that will flow through the inhaler and the air channels, which also constitutes the powder chambers and contains preloaded dose(s) of powder, be sealed by the same tape. Thus, when the tape is removed, all the features of the disposable inhaler are made accessible; the inlet and outlet holes and the air channels containing the powder. Since the freeing of air channels are performed in this unique way the number of parts are reduced to a minimum while maintaining safety and good usability. The design eliminates the risk of contamination of the air channels and thus the disposable inhaler does not need a protective bag or similar around it, thereby reducing costs while maintaining a simple way of using the disposable inhaler.

A further advantage is the upward oriented outlet holes. The user inserts the inhaler until it reaches or is near the tongue. At this position, the outlet holes are correctly positioned to make the air flowing from the outlet holes will miss the tongue and instead pass into the space that exists between the tongue and the palate. The airflow direction out of the outlet holes are affected, among other things, by the placement of the holes, upwardly, combined with that the air hits a canted end walls. But through the momentum of the airflow, the airstream is essentially directed horizontally and streams into the oral cavity. The exact angle from the outlet holes is of less significance. The crucial part is that the air, when it comes out of the inhaler nozzle, does not hit the tongue directly. The upward outlet holes then allows the initial airstream, to not hit the tongue which happens to inhalers having an outlet directed straight backwards and into the user's mouth. The air channel formed by the tongue and palate in the mouth is essentially targeted about 45 degrees upward in its first part, which is about 20 mm long. The airflow speed is at its greatest when it leaves the inhaler nozzle. The nozzles' narrow section provides high air velocity since, the many times larger cross-section in the oral cavity, reduces the speed. For inhalers with straight outlet nozzles, the tongue meets the air flow when the speed is at its greatest. By inhalation of medicinal powder, which has a much higher density than the air, the powder will, because of its mass inertia, partially continue straight ahead and get caught on the mucous membrane. This happens with inhalers that have an outlet directed straight backwards and into the user's mouth. The powder that gets stuck in the oral cavity follows the saliva down to the stomach. The therapeutic effect is thereby lost for the percentage of the powder that follows the saliva down into the stomach. The present invention directs the air approximately 45 degrees upward. Hence the upward angled outlet holes reduce the amount of powder that sticks to the tongue. Two of the patents mentioned in the chapter “technical background” display other ways of addressing this problem. For example, you can increase the amount of powder. The driving force to avoid spills onto the tongue is that the medical powder is expensive; if you can reduce the amount of powder the product becomes cheaper.

An alternative construction would be for the outlet holes to be punched or cut in the shape of a semi-circle and form flaps that affects the direction of the airflow. When inhalation is ongoing the air stream forces the flaps upward and the foils balanced combination of thickness and stiffness can be chosen so that it affects the airflow angle upward within normal inhalation speeds. The flaps will allow for a more accurate directed air stream to a 30 to 60 degree angle, preferably 45 degrees, that is, in a direction that is consistent with the gap formed between the tongue and palate.

Another optional construction is to arrange a flap having two or more parts at the exit hole i.e. one first part that extends out over approximately half of the hole from the side of the hole that is closest to the intake hole and another second part that extends out over the exit hole from the opposite side of said hole and hence meets the other flap about half way over the hole. In this scenario the second flap part will, when inhalation is ongoing be forced to flap upwards by the air stream. The foils balanced combination of thickness and stiffness can be chosen so that it affects the airflow angle, within normal inhalation speeds, to an even more upward angle then given with just one flap part at the exit hole. Various other ways of designing said flap can be made. The flap can have its fastening point at the far end of the exit hole i.e. closest to the inside of the mouth. The flap over the exit hole can be punched out as an X or similar. Another optional construction is that a flap is arranged at the inlet hole. The purpose of this flap is to prevent powder from falling out if the user is incorrectly positioning the disposable inhaler, for example vertically or upside-down. The flap forces the powder to remain left in the single dos disposable inhalers air channel. When the user inhales a negative pressure is created in the air channel, the flap at the inlet hole is folded down and an opening for the airstream is created.

Another option is to arrange a text on the top of the inhaler, which instructs the user to “place the index finger at a specific point for inhalation”. This will then help the user to insert the inhaler in the oral cavity to a correct and favorable position. When the index finger touches the upper lip the outlet holes are in a suitable position to let the escaping air from the outlet holes to pass into the space that exists between the tongue and palate in an optimal manner.

Yet another option is to arrange an elevation or evisceration on the top of the inhaler and instruct the user to position the index finger at the said elevation/evisceration. The elevation/evisceration may for example consist of two parts that are so arranged that there is room for the width of an index finger between them. When the index finger touches the upper lip the outlet holes are in a suitable position to let the escaping air from the outlet holes to pass into the space that exists between the tongue and palate in an optimal manner.

Yet another option is to arrange an elevation/evisceration on top or on the underside of the inhaler and instruct the user to put the teeth in the said elevation/evisceration. The teeth have the advantage that they are arranged in a precise location in relation to the space that exists between the tongue and palate. The elevation/evisceration may for example consist of two upward pointing elevations on top of the inhaler with some space between them to fit against the maxillary incisors. When the front teeth are located between the two humps the outlet holes are precisely positioned to let the escaping air from the outlet holes to pass into the space that exists between the tongue and palate in an optimal manner.

Of course it is also possible to arrange only one elevation as indication on where the upper teeth should be placed. The inhaler is then passed into the mouth so far that elevation reaches the upper teeth, after which the user can inhale the doses of powder.

Alternatively, the elevations, or only one elevation can be arranged on the under-side of the inhaler to indicate where in the under teeth must be placed at inhalation.

It is also possible that instead of elevations arrange eviscerations in the inhaler to indicate where the index finger, thumb, upper teeth or the lower teeth shall be positioned at inhalation.

Another alternative form is that the covering foil is provided with an inlet hole and an outlet hole per air channel instead of a larger hole covering both air channels in the two channel variant of the invention.

The medical powder must be protected against moisture. Moisture-absorption makes the powder stick together in clumps that can cause coughing. The powder can get stuck together so that inhalation is made difficult or impossible. The present invention is built like a blister. Several multidose-inhalers are based on the principle that a blister in the form of a band with for example 60 doses are fed one on one to an opening mechanism rips or punctures the depressions containing the powder.

For moisture protection for the inventive inhaler is a thermoformed transparent mold-body in a plastic wrap, with a 0.3 mm thick aluminum layer, 6-8 microns, or a pressed frame completely in aluminum with a material thickness approximately 0.15 mm. The mold-body can alternatively be manufactured as a solid unit where the air channels are formed to a cavity, for example by means of mechanical machining, injection molding, cold pressing, hot pressing or die-casting. In the area of the cavity the mold-body is covered with a foil with inlet and outlet holes. The material is selected preferably for low permeation; COC (Cyclic Olefin Copolymer) is a material with good performance. The foil is covered by an adhesive tape in the area of the air channel. The tape consists of a plastic wrap with an aluminum layer, 6-8 microns. Aluminum is the main moisture barrier in construction. The three parts are joined together with heat. Binders are PVC and the joints should not be thicker than 30 microns, the seal length should be at least 3 mm. the tape may be self-adhesive as an alternative to heat-adhesive. With these conditions the regulatory requirements for moisture protection are met. It is an advantage that the innovative disposable inhaler can be constructed in this manner since the efficiency of the moisture protection is always a difficult issue for inhalers. The said construction is a well proven technology for moisture protection which reduces development and testing costs. In the present invention the mold-body is placed on the underside of the inhaler but it is of course also possible to organize form the body on top of the inhaler.

The described disposable inhaler is considerably cheaper, smaller and thinner than the famous disposable inhalers including the one variant of the invention that has at one powder chamber. The present invention consists of cheaper parts than the known disposable inhalers and accomplishes this with no loss of safety combined with an easy operation as well as displaying a variant that can store at least two different medical powders separately until inhalation.

The above mentioned and more purposes and benefits are achieved by the invention using a disposable inhaler in accordance with, in the characteristic part of the patent claim 1 specified features.

### BRIEF LIST OF THE DRAWINGS

The invention is described in more detail below in a few preferred design examples with use of the attached drawings.

FIG. 1 displays an inventive disposable inhaler in an exploded view.

FIG. 2A-2B displays an inventive disposable inhaler, and how a tape is withdrawn and how that exposes the inlet and outlet holes.

FIG. 3A-3B displays an inventive disposable inhaler and how the airstream transports the substance in powder form via the air channel.

FIG. 4 displays an inventive disposable inhaler and that the inlet holes are shaped as flaps.

FIG. 5A displays an inventive disposable inhaler and two elevations for positioning of the index finger at inhalation.

FIG. 5B displays an inventive disposable inhaler and two elevations for the placing of the incisors at inhalation.

FIG. 6A, 6B and 6C displays an inventive single dose disposable inhaler in an exploded view.

FIG. 7A and 7B displays an inventive single dose disposable inhaler, how a tape is withdrawn and how that exposes the inlet and outlet holes.

FIG. 8A and 8B displays an inventive single dose disposable inhaler and how the airstream transports the substance in powder form via the air channel.

FIG. 9 displays an inventive single dose disposable inhaler and that the inlet hole is shaped as a flap.

FIG. 10A displays an inventive single dose disposable inhaler and two elevations for positioning of the index finger at inhalation.

FIG. 10B displays an inventive single dose disposable inhaler and two elevations for the placing of the incisors at inhalation.

FIG. 11 illustrates a disposable inhaler with two flap parts at the exit hole helping the airflow and hence the powder to exit the disposable inhaler into a steeper angle than given with only one flap.

FIG. 12A, 12B and 12C illustrates different ways of designing the flap of the exit hole to help increasing the angle of the airstream.

### DESCRIPTION OF PREFERRED EMBODIMENTS

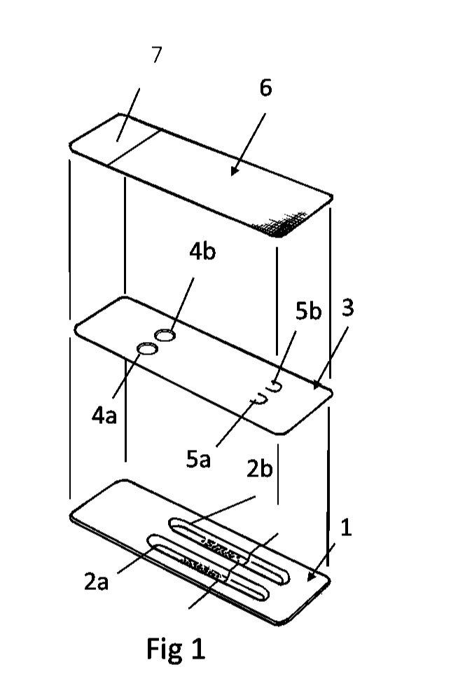

FIG. 1 Displays in an exploded view an inventive disposable inhaler consisting of a mold-body 1 made of a rigid or semi-rigid material fitted with recesses which forms a first and second air channel 2a,b containing, for example, a preloaded amount of medical substance in powder form. The mold-body 1 is covered by a foil 3 which can be transparent. The foil 3 is equipped with inlet holes 4a,b and outlet holes 5a,b at each end of the air channels 2a,and b. A tape 6 seals the inlet and outlet holes as well as it seals the air channels 2a,b. One end of the tape 7 has no adhesive agent, in order to facilitate the user to take hold of the tape when it shall be removed.

The mold-body can alternatively be manufactured as a solid unit where the air channels are cavities, for example by injection molding, cold pressing, hot pressing or die-casting.

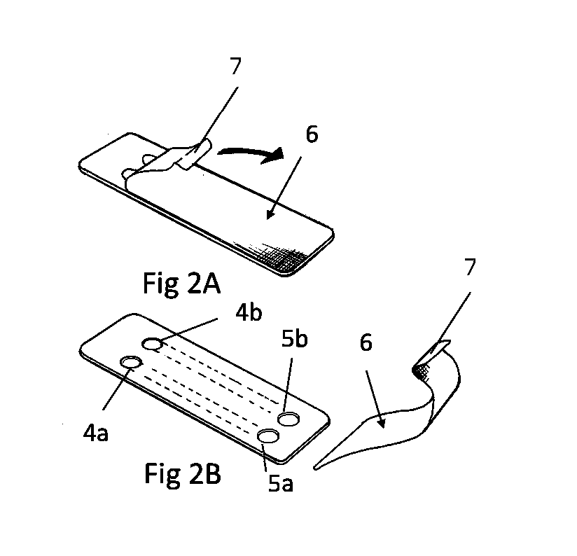

FIG. 2A and 2B displays how the tape 6 can be removed and how the inlet holes 4a,b and outlet holes 5a,b are exposed when the tape is fully removed. The air channels 2a,b are protected from foreign particles as long as the tape has not been removed. The air channels are also moisture protected until the tape is removed.

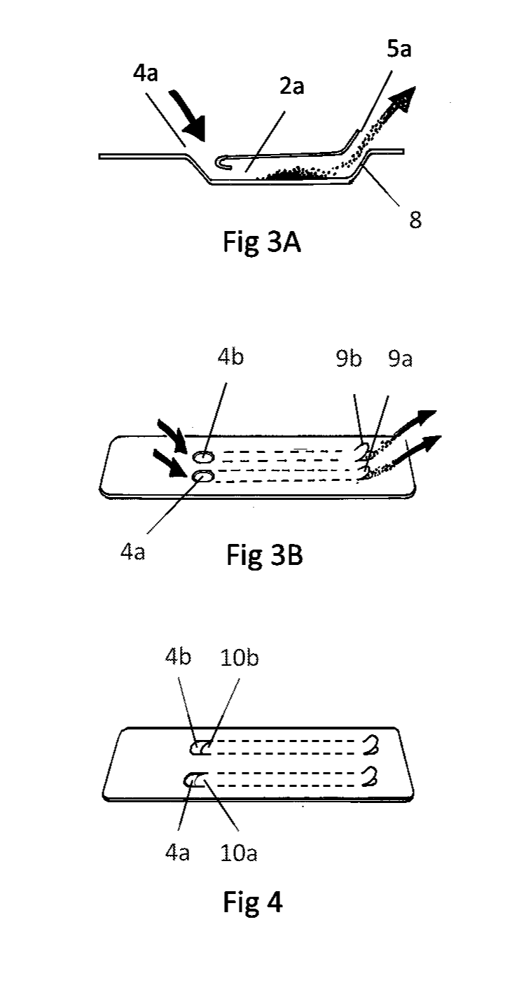

FIG. 3A is a cross section from the side of the air channel 2a and displays how the air stream passes through the inlet hole 4a, through the recess that, at inhalation, forms the air channel 2a and out through the outlet hole 5a. The air stream is driven by the under-pressure formed by the user's inhalation. The direction of the airflow out of the outlet hole 5a is a combination placement of the hole and its direction upwards, with a canted end-wall 8 in the air channel 2a and combined with the momentum/current direction of the air which is directed horizontally inward toward the oral cavity.

FIG. 3B displays how the outlet holes 5a,b form flaps 9a,b which affects the airflow direction. When the inhalation is started the air flow forces the flaps to open upwards and the property of the foil, a combination of thickness and stiffness, is selected so that upward-angle of the airflows are affected by normal flows of inhalation. The flaps will allow a more accurate direction of the airstreams preferably in 30 to 60 degrees, especially about 45 degrees, a direction that is consistent with the initial gap formed between the tongue and palate of a user.

FIG. 4 displays an inventive disposable inhaler and that the inlet holes 4a,b is designed as flaps 10a,b in order to prevent the powder from falling out if the user positions the disposable inhaler incorrectly.

When the user inhales an under-pressure is created at the inlet holes 10a,b and the flaps folds downwards, an opening for the airstreams are created.

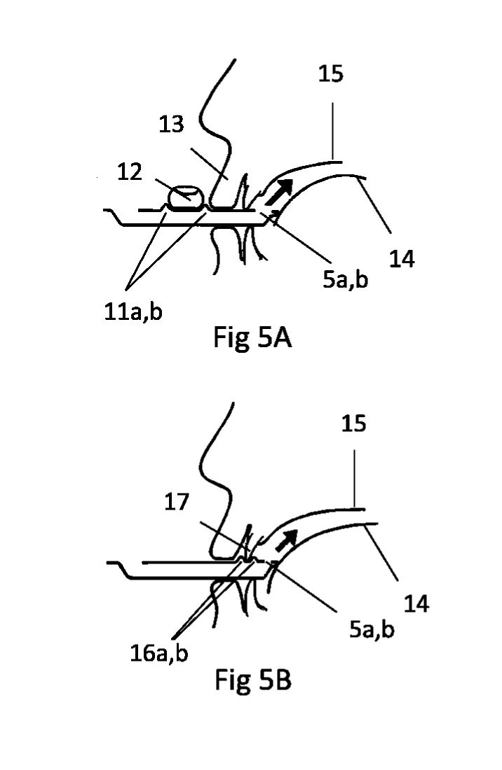

FIG. 5A displays an inventive disposable inhaler with two elevations 11a,b on the top of the inhaler. The elevations 11a,b indicates where an index finger should be placed at inhalation. The inhaler is then inserted into the mouth with the index finger positioned between the two elevations 11a,b. When the index finger 12 touches the upper lip 13 the outlet holes 5a,b are optimally positioned to let the escaping air from the outlet holes 5a,b to pass into the space that exists between the tongue 14 and palate 15.

Of course it is also possible to arrange one or several elevations on the underside of the inhaler. The elevations (not shown) indicate, for example, where a thumb is to be positioned at inhalation. The thumb is naturally placed on the underside of the inhaler in line with the index finger on the top. The inhaler is then inserted into the mouth until the thumb reaches the lower lip, after which the user can inhale the powder doses.

FIG. 5B displays an inventive disposable inhaler and that two elevations 16a,b indicates where the front teeth 17, shall be positioned at inhalation. The inhaler is inserted in the mouth until the front teeth are positioned between the two elevations. In this position, the discharge holes 5a,b are optimally positioned to let the escaping air from the outlet holes to pass into the space that exists between the tongue 14 and palate 15.

Of course it is also possible to arrange only one elevation 11a. The inhaler is then inserted into the mouth so that the elevation 11a reaches the front teeth, after which the user can inhale the powder doses.

It is also possible to instead of elevations arrange one or several eviscerations/recesses in the inhaler to indicate where the index finger, thumb, incisors or the teeth in the lower jaw shall be placed on the inhaler at inhalation (not shown).

As described earlier another embodiment can be a single dose disposable inhaler i.e. the same concept as described in FIGS. 1-5 but with only one recess instead of two (or more).

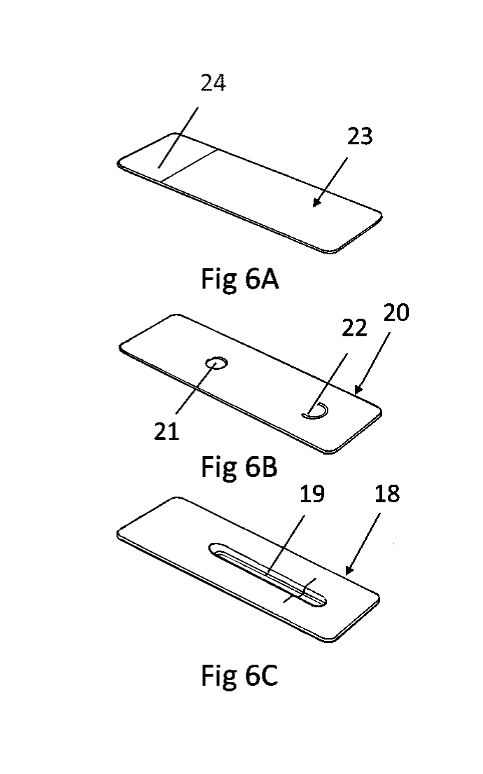

FIG. 6 A-6C displays in an exploded view of an inventive single dose disposable inhaler consisting of a mold-body 18 made of a rigid or semi-rigid material fitted with a recess which forms an air channel 19 containing, for example, a preloaded amount of medical substance in powder form. The mold-body 1 is covered by a foil 20 which can be transparent. The foil 20 is equipped with an inlet 21 and an outlet hole 22 at each end of the air channel 19. A tape 23 seals the inlet and outlet holes as well as it seals air channel 19. One end of the tape 24 has no adhesive agent, in order to facilitate the user to take hold of the tape when it shall be removed.

The mold-body can alternatively be manufactured as a solid unit where the air channel is a cavity, for example by injection molding, cold pressing, hot pressing or die-casting.

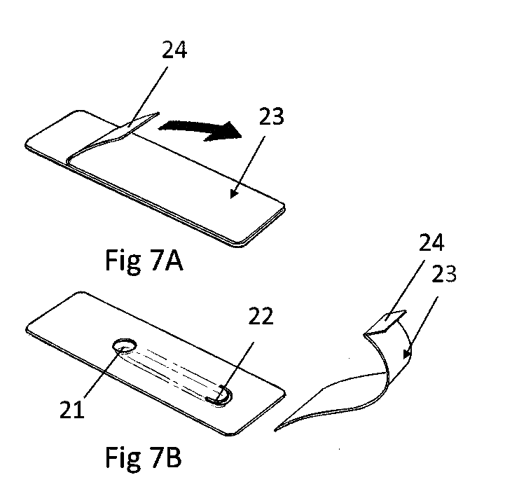

FIG. 7A and 7B displays how the tape 23 is initially removed and how the inlet 21 and outlet 22 holes are exposed when the tape is fully removed. The air channel 19 is protected from foreign particles as long as the tape has not been removed. The air channel is also moisture protected until the tape is removed.

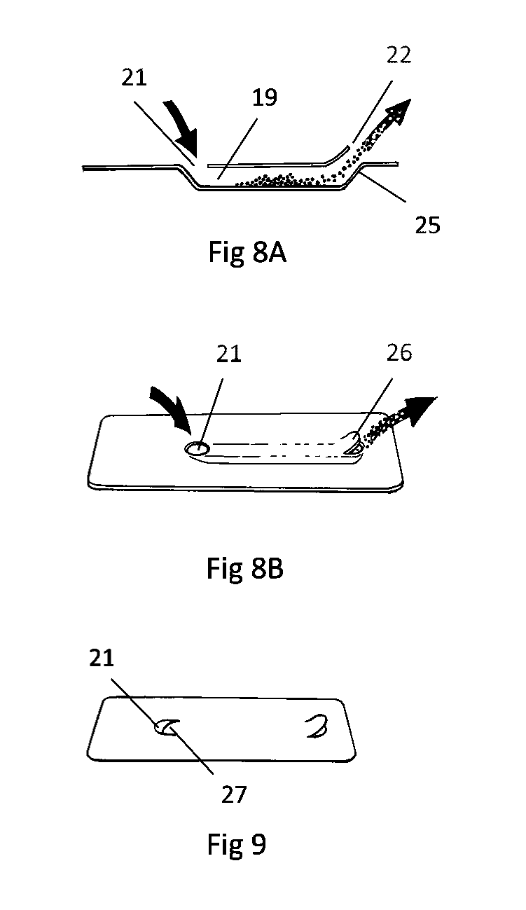

FIG. 8A is a cross section from the side of the air channel and displays how the air stream passes through the inlet hole 21, through the recess that, at inhalation, forms the air channel 19 and out through the outlet hole 5. The air stream is driven by the under-pressure formed by the user's inhalation. The direction of the airflow out of the outlet hole is a combination placement of the hole and its direction upwards, with a canted end-wall 25 in the air channel and combined with the momentum/current direction of the air which is directed horizontally inward toward the oral cavity.

FIG. 8B displays how the outlet hole 22 forms flap 26 which affects the airflow direction. When the inhalation is started the air flow forces the flap to open upwards and the property of the foil, a combination of thickness and stiffness, is selected so that upward-angle of the airflow is affected by normal flows of inhalation. The flap will allow a more accurate direction of the airstream preferably in 30 to 60 degrees, especially about 45 degrees, a direction that is consistent with the initial gap formed between the tongue and palate of a user.

FIG. 9 displays an inventive single dose disposable inhaler and that the inlet hole 21 is designed as a flap 27 in order to prevent the powder from falling out if the user positions single dose disposable inhaler incorrectly. When the user inhales an under-pressure is created at the inlet hole and the flap folds downwards, an opening for the airstream is created.

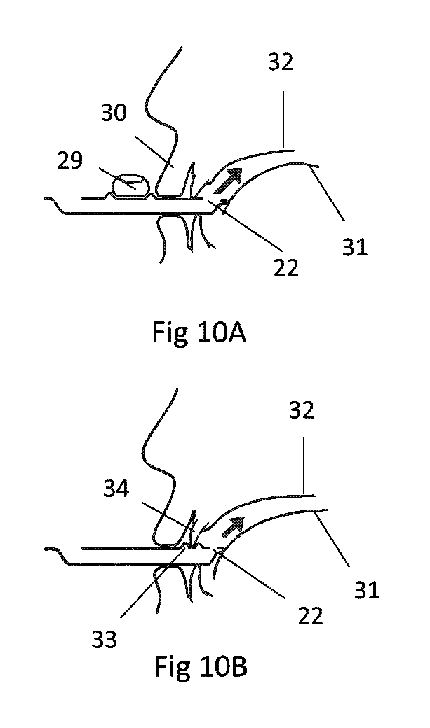

FIG. 10A displays an inventive single dos disposable inhaler with two elevations 28 on the top of the inhaler. The elevations indicate where an index finger should be placed at inhalation. The inhaler is inserted with the index finger positioned between the two elevations. When the index finger 29 touches the upper lip 30 the outlet hole 22 is optimally positioned to let the escaping air from the outlet hole 22 to pass into the space that exists between the tongue 31 and palate 32.

Of course it is also possible to arrange an elevation on the underside of the inhaler. The elevation indicates, for example, where a thumb is to be positioned at inhalation. The thumb is naturally placed at the elevation on the underside of the inhaler in line with the index finger on the top. The inhaler is then inserted into the mouth until the thumb reaches the lower lip, after which the user can inhale the powder dose.

FIG. 108 displays an inventive single dos disposable inhaler and two elevations 28 that indicates where the front teeth 16, shall be positioned at inhalation. The inhaler is inserted in the mouth until the front teeth are positioned between the two elevations. In this position, the discharge hole 5 is optimally positioned to let the escaping air from the outlet hole 5 to pass into the space that exists between the tongue 31 and palate 32.

Of course it is also possible to arrange only one elevation 28. The inhaler is then inserted into the mouth so that the elevation 28 reaches the front teeth, after which the user can inhale the dose of powder.

Alternatively, the elevations, or only one elevation be arranged on the underside of the inhaler to indicate where in the teeth must be placed at inhalation.

It is also possible to instead of elevations arrange eviscerations in the inhaler to indicate where the index finger, thumb, incisors or the teeth in the lower jaw shall be placed on the inhaler at inhalation.

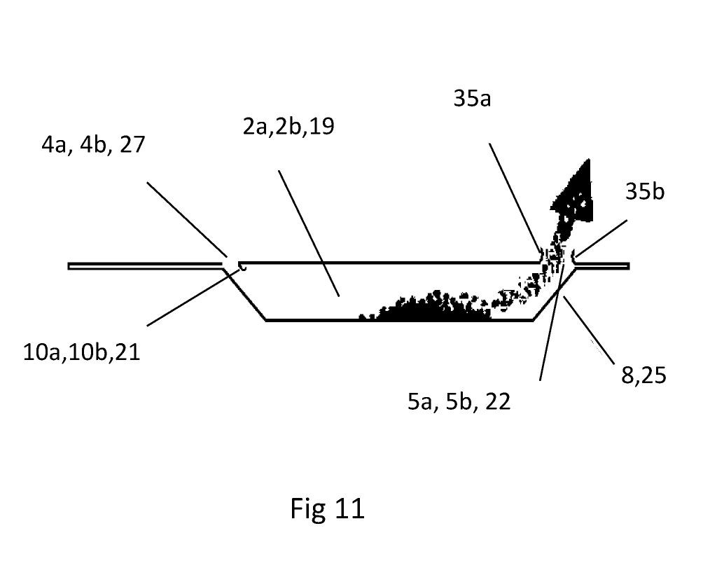

FIG. 11 is a cross section from the side of the air channel 25 and illustrates how the air stream passes through the inlet hole 21, through the recess that, at inhalation, forms the air channel 19 and out through the outlet hole 22. The air stream is driven by the under-pressure formed by the user's inhalation. The direction of the airflow out of the outlet hole 22 is a combination placement of the hole and its direction upwards, with a canted end-wall 25 in end of the air channel 19 and combined with the momentum/current direction of the air which is directed horizontally inward toward the oral cavity. The outlet hole is here covered by a split flap 35a,b. The second flap part 35b seen in this figure will further help to angle the airstream upwards.

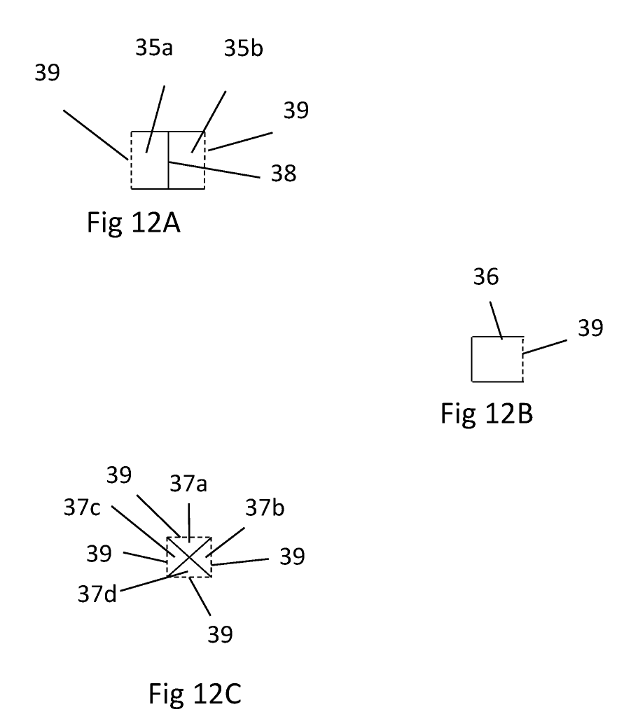

FIG. 12A to C illustrates different possible ways to design or punch the flap 35 and locate it in different positions at the outlet hole. The direction of the airflow is from left to right in the FIGS. 11 and 12A to C.

The flap 35 at the outlet hole can advantageously be designed as a split flap as illustrated in FIGS. 11, 12A and C, The flap 35 and 37 consists here of two or more parts which all will fold upwards upon inhalation but where at least one part 35b, 37b is located at the far edge of the outlet hole 5a,b, 22, downstream the outlet hole 5a,b, 22 seen in the air flow direction and again arranged to influence the air stream to flow substantially straight upwards from the air channel 19 and into the user's mouth.

The flap parts 35a,b may be of similar size as illustrated in FIG. 12A, i.e. the division line 38 can be located in the middle of the flap 35 but the division line can of course also be located closer to the front or distal edge of the outlet hole 5a,b, 22, seen in the direction of the air flow so that one flap part 35a,b is larger than the other, in order to influence the air to flow in a desired manner, i.e. straight upwards or askew up from the air channel 19 and the outlet hole 5a,b, 22.

FIG. 12B shows how the flap 35 so-called hinge 39 or bend line, about which the flap 35 is folded up, is located on the opposite side, i.e. downstream of the outlet hole 5a,b, 22, i.e. to the right of the air channel 19 thereby forcing the flap 35b to bend upwards by the air stream. Then the air flow even more is affected to flow upward at a steeper angle at inhalation whereby the quantities of medical powder that risk to meet and adhere to the tongues front/outer portion substantially is reduced.

The flaps 35,36 and 37 and their parts may have different shapes as shown e.g. in FIG. 12C where the flap 37 consists of four parts 37a-d cut or punched out in a shape of a cross.

The material of the flaps 35, 36 and 37 or their flap parts are selected or designed to have such rigidity that it/they fold up enough but no more in order to control the air flow substantially upwards or in a steep askew desired direction.

These optional designs of the flap described in FIGS. 11 and 12A to C, covering the outlet hole 5a,b, 22, can be made regardless of the amount of outlet holes 5a,b, 22 i.e. thus also it could be used in the earlier described double dose (or more) disposable inhaler.

The description above is primarily intended to facilitate the understanding of the invention, and is of course not limited to the presented embodiments, also other embodiments of the invention are possible and conceivable within the framework of the innovative thought and the subsequent claims and scope of protection. Hence the disposable inhaler can be fitted, as described earlier, with one or more air channels with associated inlet and outlet holes.

1. An inhaler for single use and for substances in powder form, the inhaler comprising:
an elongated shape body arranged to at least partially be inserted into the user's mouth, comprising:
at least one air channel, preferably two or more, arranged in the body, that a foil is provided with inlet holes and outlet holes adjacent to the air channel, a substance in powder form is placed in the air channel, that a tape is arranged, in the inhaler storage mode/position, to cover the inlet and the outlet holes of the foil and thereby preventing the substance in powder form to leave the inhaler air channels, and that the tape is being removable before the inhaler use and when removed exposes both the inhaler inlet and outlet holes and thus enables the substance in powder form, leaving the air channel by means of the air flow that occurs when the user inhales, through the inhaler air channel,
wherein each air channel is provided with an inclined/canted end wall at or near the outlet hole, in that said inclined/canted end wall angles the air flow out of the air channel sloping upwards at the outlet.

2. The inhaler according to claim 1,
wherein the inlet hole and/or the outlet hole in the inactive position, when no air flows through the air channel, is covered by deflectable flaps provided in the foil.

3. The inhaler according to claim 2,
wherein at least the flap at the outlet hole is formed/stamped essentially like a semicircle.

4. The inhaler according to claim 2,
wherein the foils thickness/stiffness is so arranged as to affect the angle of the air stream out of the outlet hole so that the flap for inhalation is folded up at an angle of between 30 to 60 degrees relative to the foils main surface.

5. The inhaler according to claim 2,
wherein the flap is adapted to be folded upwards at inhalation directing the airflow upwards at an angle of preferably about 45 degrees angle to the foils main surface.

6. The inhaler according to claim 2,
wherein the inclined/canted end wall at or near the outlet hole and the flexible flap are arranged so that they in open position together form an angle that directs the air flow in an upward angle of between 30 and 60 degrees, preferably about 45 degrees, relative to the air channels principal direction, and into the natural gap formed between the tongue and palate in human beings.

7. The inhaler according to claim 1,
wherein a flap is provided at each inlet hole for preventing powder falling out during use.

8. The inhaler according to claim 7,
wherein the flap at the inlet hole is arranged to flex downwardly at inhalation.

9. The inhaler according to claim 1,
wherein the foil is transparent and allows the powder in the air channel to be visible to the user.

10. The inhaler according to claim 1,
wherein the inhaler is provided with at least one elevation or immersion/withdrawal that indicates how far the user must insert the inhaler in his mouth.

11. The inhaler according to claim 2,
wherein at least one part of a flap at the outlet hole is arranged downstream of the outlet hole and adapted to control the air flow substantially upwards from the air channel.

12. The inhaler according to claim 11,
wherein the flap comprises at least two flap parts.

13. The inhaler according to claim 11,
wherein the material of the flap or the flap parts are selected/designed so that the flap or the flap parts has such a stiffness that they are folded upwardly enough, but not more, for controlling the air flow from the air channel substantially straight upwards from the air channel.
